SEQUENCE LISTING (23 sequences)

SEQ ID NO: 1 - protein, 359 aa, Sulfolobus tokodaii
MILMRRTLKAAILGATGLVGIEYVRMLSNHPYIKPAYLAGKGSVGKPYGEVVRWQTVGQVPKEIADMEIKPTDPKLMDDVDIIFSPLPQGAAGPVEEQFAKEGFPVISNSPDHRFDPDVPLLVPELNPHTISLIDEQRKRREWKGFIVTTPLCTAQGAAIPLGAIFKDYKMDGAFITTIQSLSGAGYPGIPSLDVVDNILPLGDGYDAKTIKEIFRILSEVKRNVDEPKLEDVSLAATTHRIATIHGHYEVLYVSFKEETAAEKVKETLENFRGEPQDLKLPTAPSKPIIVMNEDTRPQVYFDRWAGDIPGMSVVVGRLKQVNKRMIRLVSLIHNTVRGAAGGGILAAELLVEKGYIEK

SEQ ID NO: 2 - protein, 357 aa, Metallosphaera sedula
MRRTLKAAILGATGLVGIEYVRMLADHPYIKPTYLAGKGSVGKPYGEIVRWQTVGNVPKEVANQEVKPTDPKLMDDVDIIFSPLPQGAAGPVEEQFAKLGFNVISNSPDHRFDMDVPMIIPEVNPHTVTLIDEQRKRRDWKGFIVTTPLCTAQGAAIPLTPIYQNFKMSGVMITTMQSLSGAGYPGIASLDIVDNALPLGDGYDAKTVKEITRILSEVKRNVQEPGVNEITLDATTHRIATIHGHYEVAYVTFKEDTDVRKVMESMESFKGEPQDLKLPTAPEKPIIVTTQDARPQVFFDRWAGNPPGMSVVVGRLKQVNPRTIRFVSLIHNTVRGAAGGGVLTAELLVEKGYIDKR

SEQ ID NO: 3 - protein, 350 aa, Candidatus caldiarcheum subterraneum
MKTYSVAILGATGMVGQHYIRMLYRHPWFRITALTGKESVGRKYVEAVRGEAPEPPKEIAEMEVLPTDPKKVDADFVFSCLPTEAAREAEPKFAEAGFPVFSDAAAYRMEEDVPLIVPEINHDHLNMVHIQRKKRGWEGYIVTTPNCTTVGLVLPLQPLKQHLGVKKVIVTTMQAVSGAGYPGVASLSILGNVIPYISGEERKVETETAKILGRYGDGRFTHDSVEVHATCTRVPTLDGHMESIYLETAKPADEETVAELLAEYVSLPQELNLPTAPARPIVVRRELDRPQTRIDVDAGTVPGMSVSVGRIRVNGEKVRFISLSHNLIRGAAGGTILTAELARHMGLLGE

SEQ ID NO: 4 - protein, 357 aa, Sulfolobales archaeon Acd1
MRRTLKAAILGATGLVGIEYVRMLSQHPYIKPAYLAGKGSVGKAYSEVVRWQTVGQVPKEVADMPVLPTDVNEIKKAGVDIVFSPLPQGAAGPVEEEFAKAGFPVISNSPDHRFDPDVPLMIPEVNGHTASLIDEQKKRRDWSGFIVTTPLCTAQGIAIPLAPIYRDFRVDSVFITTMQSLSGEGYPGVASLDVVDNIKVLGDAYDAKTVKEVTRILSEVKRNVPGTMDELTLSATTHRIATIHGHYEVMYVTFKEDVKVEKVKETLANFKGEPQDMKLPTAPSRPILITELDNRPQPYFDRWAGDVPGMSVVVGRLKQVNNRTVRLVSLIHNTVRGAAGGGILVAEYLIEKGYIPK

SEQ ID NO: 5 - protein, 354 aa, Sulfolobus acidocaldarius Ron12/I
MRRVLKAAILGSTGLVGIEYVRMLANHPYIKVAYLAGKGSVGKPYGEVVRWQTIGQIPKEVANMEIKPTDPKLMDDVDLVFSPLPAGAAGPVEEEFAKHGFKVISDSPDHRFEPDIPLLIPEINPHTITLIDEQRKKRDWKGFIVTTPLCAAQGVLLPLAPIYQNFKVDSVFITTMQAVSGEGYPGVASLDIIDNIKVLGENYDNKLIKEVHRVLSETKRNVNDSGNDVTLSATTHRVATIHGHYEIIYVTFKEDVNVEKVREAMDNFKGEPQNLKLPTAPSKPIILTNEDSRPQVYFDRWAGEIPGMSVVVGRLSQVNRRAIRFASLIHNTVRGAAGGGILATEFLVEKGYMD

SEQ ID NO: 6 - protein, 352 aa, Sulfolobus acidocaldarius Ron12/I
MRRVYKAAILGSTGLVGIEYVRMLANHPYIKPTYLAGRGSVGKPYGEVVRWQTIGQIPKEIANQEIRPTDPKQMDDVDLVFSPLPAGSAAQVEDEFAKLGFKVISNSPDHRLEPDIPLIIPEVNPHSLNLIEEQKKRRDWEGFIVTTPLCTAQGVLIPLVPIYQNFRVQSVFITTMQALSGAGYPGVASLDVIDNILPLGNEYDAKMVKEMTKVLNSTKRNVSDESNINISTTTHRVPTIHGHYAVVYVTFKENVDLGKIRESLVNFSGEPQALKLPTAPEKVIVLTEQDNRPQVYFDRWLGDPPGMSVIVGRLTQVDNNAIRFVSLIHNSVRGAAGGGILTAELLINRKYI

SEQ ID NO: 7 - protein, 553 aa, Metallosphaera sedula
MVTPERVKEWESKYLQPWISKRKERKNKFTTPSGIEIKTLYTPLDLKGDYEEKIGFPGEYPYTRGIYPNMYRGRIWTIRQYAGFGSAEDTNARFRKLLEAGQTGLSTAFDLPTQLGLDPDNELAYTEVGVVGVSMFHWKEMDIVTNQIPLNKVSTSMTINATAMELLSMYVATAESRGVSPTEIDGTVQNDILKEYIARKNYIYPPEPSMRYAIDIIEYSYKNIPKWHPISISGYHIREAGADAVLEVAFTLADGIEYVRRTAERGIPVDDFAPTLSFFFAGYTNLFEEVAKFRAARRMWAKIMRDMFNAKKADSMTLKFHTQTGGAELTAQQPEINIIRTTIQALAAALGGTQSLHVNSYDEAVALPSEKAAKIAIRVQQIVAYESGSTETVDPLAGSYYVEWLTDEIEERAWKIIERVEGMGGMMKAVERGFPQAEIAESAYRLQKKIEEGEMIRVGVNMSYEPDWIGTTEVFRVNPEIRERVLTRLKKYRSERDQMKVRDSLNALRKAAENPSVNLFPYVLDAIKKGCTVGEISSTLREIWGEYKEPIIF

SEQ ID NO: 8 - protein, 155 aa, Metallosphaera sedula
MREYLNYLNLRDMILLMDKRIKVVVAKLGLDGHDRGAKVIARALKDAGMEVVYTGLRQTPEQIVRTAIQEDADVIGISILSGAHLELMPKIVEALKKAGLDDVGLVLGGVIPPEDIPKLKAMGVDDVFLPGTSLKEIAQRVSKLASTKRGIKVEG

SEQ ID NO: 9 - protein, 140 aa, Metallosphaera sedula
METLDIDHVGVAVENLEEAIKLYTEKMGMKLVHREDLPDRGIKVAFLTGNEGTTAVELMEPMNHEDPNNTVAKFLKTRGQGMHHLAVKVKDINSSLRDLEGKGLTLIDKNGRKGARGHLVAFVHPKSVMGLLLELVQETH

SEQ ID NO: 10 - protein, 295 aa, Pseudomonas putida
MRIAFIGLGNMGAPMARNLIKAGHQLNLFDLNKTVLAELAELGGQISPSPKDAAANSELVITMLPAAAHVRSVYLNDDGVLAGIRPGTPTVDCSTIDPQTARDVSKAAAAKGVDMGDAPVSGGTGGAAAGTLTFMVGASAELFASLKPVLEQMGRNIVHCGEVGTGQIAKICNNLLLGISMIGVSEAMALGNALGIDTKVLAGIINSSTGRCWSSDTYNPWPGIIETAPASRGYTGGFGAELMLKDLGLATEAARQAHQPVILGAVAQQLYQAMSLRGEGGKDFSAIVEGYRKKD

SEQ ID NO: 11 - DNA, 26 bp, Artificial Sequence
  MCRstF Primer
atgagctcatgagaagaactttgaaa

SEQ ID NO: 12 - DNA, 26 bp, Artificial Sequence
  MCRstR Primer
ttctcgagttacttttcgatgtaacc

SEQ ID NO: 13 - DNA, 26 bp, Artificial Sequence
  MCRmsF Primer
atgagctcatgagaagaactttgaaa

SEQ ID NO: 14 - DNA, 26 bp, Artificial Sequence
  MCRmsR Primer
ttctcgagttatctcttatcaatgta

SEQ ID NO: 15 - DNA, 26 bp, Artificial Sequence
  ASDccF Primer
atgagctcatgaaaacttactccgtc

SEQ ID NO: 16 - DNA, 26 bp, Artificial Sequence
  ASDccR Primer
ttctcgagtcattcgcctaacaacca

SEQ ID NO: 17 - DNA, 26 bp, Artificial Sequence
  ASDsarF Primer
atgagctcatgagaagaactttgaag

SEQ ID NO: 18 - DNA, 26 bp, Artificial Sequence
  ASDsarR Primer
ttctcgagttacttagggatgtaacc

SEQ ID NO: 19 - DNA, 26 bp, Artificial Sequence
  ASDsac1F Primer
atgacgtcatgagaagagtcttgaaa

SEQ ID NO: 20 - DNA, 26 bp, Artificial Sequence
  ASDsac1R Primer
ttctcgagtcaatccatgtaaccctt

SEQ ID NO: 21 - DNA, 26 bp, Artificial Sequence
  ASDsac2F Primer
atgagctcatgagaagagtttacaaa

SEQ ID NO: 22 - DNA, 26 bp, Artificial Sequence
  ASDsac2R Primer
ttctcgagtcagatgtactttctgtt

SEQ ID NO: 23 - protein, 44 aa, Artificial Sequence
  Conserved Domain between ASDsac1 and ASDsar
IKVLGDAYDAKTVKEVTRILSEVKRNVPGTMDELTLSATTHRIA

(19) United States
(12) Patent Application Publication
(10) Pub. No.: US 2017/0016033 A1
(43) Pub. Date: Jan. 19, 2017

(54) Mutant Microorganism Comprising Gene Encoding Methylmalonyl-CoA Reductase and Use Thereof

(71) Applicant: SK Innovation Co., Ltd., Seoul (KR)

(72) Inventors: Young Bin Seo, Seoul (KR); Ki Sung Lee, Daejeon (KR); Jae Yeon Park, Seoul (KR); Joong Min Park, Seoul (KR); Woo Chan Park, Sejong-si (KR); Jong Joo Lee, Daejeon (KR); Hye Sung Lee, Daejeon (KR); Kyeong Hyeon Kang, Daejeon (KR)

(21) Appl. No.: 15/207,953

(22) Filed: Jul. 12, 2016

(30) Foreign Application Priority Data
Jul. 13, 2015 (KR) 10-2015-0099352
Jun. 17, 2016 (KR) 10-2016-0075640

(51) Int. Cl. C12P 7/42; C12N 9/02; C12N 9/04; C12N 9/90

(52) U.S. Cl. CPC C12P 7/42; C12N 9/90; C12Y 501/99001; C12N 9/0008; C12N 9/0006; C12Y 504/99002; C12Y 101/01031; C12Y 102/01075

(57) ABSTRACT

Provided herein is a mutant microorganism containing a methylmalonyl-CoA reductase-encoding gene having an activity of converting methylmalonyl-CoA to methylmalonate semialdehyde and uses of the mutant microorganism. The mutant microorganism includes a gene encoding kingdom Archaea-derived methylmalonyl-CoA reductase.

### CROSS-REFERENCE TO RELATED APPLICATIONS

This application claims priority to Korean Patent Application Nos. 10-2015-0099352 and 10-2016-0075640, filed Jul. 13, 2015 and Jun. 17, 2016, respectively, the disclosures of which are hereby incorporated in their entirety by reference.

The Sequence Listing associated with this application is filed in electronic format via EFS-Web and is hereby incorporated by reference into the specification in its entirety. The name of the text file containing the Sequence Listing is 1603244-2_ST25.txt. The size of the text file is 31,779 bytes, and the text file was created on Jul. 1, 2016.

### TECHNICAL FIELD

The present invention relates to a mutant microorganism containing a methylmalonyl-CoA reductase-encoding gene having an activity of converting methylmalonyl-CoA to methylmalonate semialdehyde and the use of the mutant microorganism, and more particularly, to a mutant microorganism introduced with a gene encoding kingdom Archaea-derived methylmalonyl-CoA reductase and the use of the mutant microorganism.

### BACKGROUND ART

Methacrylic acid and/or methylmethacrylic acid (or methylmethacrylate) is a compound that can be used for preparation of polymers such as coatings, transparent plastics or adhesives. The development of processes for biosynthesizing methacrylic acid and the application thereof are in progress. For example, Evonik developed a process of synthesizing methylmethacrylic acid from, for example, ammonia, methane, acetone or methanol via 2-HIBA (2-hydroxybutyric acid) (esterification following dehydration of 2-HIBA).

Biological intermediates that can be converted into such methacrylic acid and/or methylmethacrylic acid (or methylmethacrylate) are known not only to be 2-HIBA but also to be itaconic acid, isobutyric acid, isobutylene, 3-HIBA and the like.

Regarding biological synthesis of methylmethacrylic acid, U.S. Pat. No. 8,865,439 discloses a pathway that biosynthesizes methylmethacrylic acid from a carbon source via 3-HIBA, and a recombinant microorganism containing a gene encoding an enzyme which is involved in the pathway.

It is known that methylmalonyl-CoA reductase is necessarily required in the biosynthesis pathway of methylmethacrylic acid in order to efficiently biosynthesize 3-HIBA, which can exhibit the highest theoretical yield, from glucose as shown in FIG. 1. However, methylmalonyl-CoA reductase is an enzyme that has not yet been in nature.

Thus, in order to construct a metabolic pathway for biosynthesis of 3-HIBA as shown in FIG. 1, screening of methylmalonyl-CoA reductase, an enzyme that converts methylmalonyl-CoA to methylmalonate semialdehyde, is most urgently required.

Under such a technical background, the present inventors have screened an enzyme, which exhibits methylmalonyl-CoA reductase activity, from among enzymes (MCR) that convert methylmalonyl-CoA to methylmalonate semialdehyde, and a gene encoding the enzyme, thereby completing the present invention.

### SUMMARY OF THE INVENTION

It is an object of the present invention to provide a mutant microorganism introduced with an MMCR (methylmalonyl-CoA reductase)-encoding gene, which has the ability to produce 3-HIBA (3-hydroxyisobutyric acid), and the use of the mutant microorganism.

To achieve the above object, the present invention provides a mutant microorganism derived from a microorganism having the ability to produce succinyl-CoA from a carbon source, wherein the mutant microorganism contains genes encoding the following enzymes and has the ability to produce 3-HIBA (3-hydroxyisobutyric acid):

(i) methylmalonyl-CoA mutase;

(ii) methylmalonyl-CoA epimerase;

(iii) methylmalonyl-CoA reductase (MMCR); and

(iv) 3-hydroxyisobutyrate dehydrogenase, wherein the enzyme of (iii) is an enzyme exhibiting methylmalonyl-CoA reductase (MMCR) activity, selected from among enzymes having malonyl-CoA reductase (MCR) activity.

The present invention also provides a method for producing 3-HIBA, comprising the steps of:

culturing the mutant microorganism of any one of claims 1 to 7 to produce 3-HIBA; and

recovering the produced 3-HIBA.

Because MMCR does not exist in nature, it was inevitable to bypass the metabolic pathway of MMCR in conventional processes of producing 3-HIBA from carbon sources, including glucose. However, according to the present invention, 3-HIBA can be produced through a short metabolic pathway by using an enzyme, which exhibits MMCR activity, selected from among enzymes having MCR activity. 3-HIBA produced according to the present invention can be used to produce MAA (methacrylic acid) and/or MMA (methylmethacrylic acid), which is advantageous in that it is environmentally friendly and cost-effective, because it does not involve toxic substances, such as HCN, CAN or formamide, unlike MAA (methacrylic acid) or MMA (methylmethacrylic acid) which has been produced by conventional chemical processes.

### BRIEF DESCRIPTION OF THE DRAWINGS

FIG. 1 shows a metabolic pathway for producing 3-HIBA from glucose.

FIG. 2 shows the results of cloning MMCR candidate genes by PCR.

FIG. 3 shows the results of inserting MMCR candidate genes into vectors by ligation.

FIG. 4 shows the results of culturing strains transformed with MMCR candidate genes and analyzing the expression level of each enzyme in the cultured strains.

FIG. 5 shows the results of measuring the titers of MMCR candidates using methylmalonyl-CoA as a reaction substrate.

FIG. 6 shows the results of MS analysis of a reaction product obtained using methylmalonyl-CoA as a reaction substrate.

FIG. 7 shows the results of analyzing cultures of 3-HIBA-producing strains by HPLC to determine the production of 3-HIBA.

### DESCRIPTION OF THE INVENTION

Unless defined otherwise, all technical and scientific terms used herein have the same meaning as commonly understood by one of ordinary skill in the art to which the invention pertains. Generally, the nomenclature used herein and the experiment methods, which will be described below, are those well known and commonly employed in the art.

In one aspect, the present invention is directed to a mutant microorganism derived from a microorganism having the ability to produce succinyl-CoA from a carbon source, wherein the mutant microorganism contains genes encoding the following enzymes and has the ability to produce 3-HIBA (3-hydroxyisobutyric acid):

(i) methylmalonyl-CoA mutase;

(ii) methylmalonyl-CoA epimerase;

(iii) methylmalonyl-CoA reductase (MMCR); and

(iv) 3-hydroxyisobutyrate dehydrogenase, wherein the enzyme of (iii) is an enzyme exhibiting methylmalonyl-CoA reductase (MMCR) activity, selected from among enzymes having malonyl-CoA reductase (MCR) activity.

In the present invention, the enzyme of (iii) is not specifically limited, as long as it exhibits methylmalonyl-CoA reductase activity. For example, the enzyme of (iii) may be either a monofunctional enzyme that converts methylmalonyl-CoA to methylmalonate semialdehyde, or a bifunctional enzyme that converts methylmalonyl-CoA to methylmalonyl semialdehyde and is also involved in a process that converts methylmalonate semialdehyde to 3-HIBA. Preferably, the enzyme of (iii) may be a monofunctional enzyme.

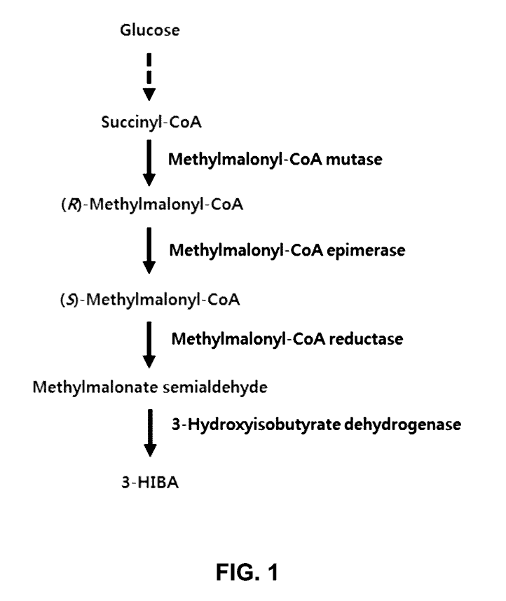

Among the enzymes, the methylmalonyl-CoA mutase (i) is involved in the conversion of succinyl-CoA, produced from the carbon source, to (R)-methylmalonyl-CoA, and the methylmalonyl-CoA epimerase (ii) is involved in the conversion of (R)-methylmalonyl-CoA to (S)-methylmalonyl-CoA. Furthermore, the methylmalonyl-CoA reductase (iii) is involved in the conversion of (S)-methylmalonyl-CoA to methylmalonate semialdehyde, and the 3-hydroxyisobutyrate dehydrogenase (iv) is involved in the conversion of methylmalonate semialdehyde to 3-HIBA. A specific pathway for synthesis of 3-HIBA is as shown in FIG. 1.

The term “3-HIBA (3-hydroxyisobutyric acid)” means a C₄-carboxylic acid, and may include an acid form (3-hydroxyisobutyric acid), a base form (3-hydroxyisobutyrate), or a mixture thereof. The term 3-HIBA may include both (R) and (S) stereoisomers. 3-HIBA may be used as an intermediate for producing MAA (methacrylic acid) and/or MMA (methylmethacrylic acid), but is not limited thereto.

Because methylmalonyl-CoA reductase, an enzyme that converts methylmalonyl-CoA to methylmalonate semialdehyde, does not exist in nature, it was inevitable to bypass the metabolic pathway of methylmalonyl-CoA reductase in conventional processes of producing 3-HIBA from carbon sources, including glucose. That is, in the conventional processes, 3-HIBA was biosynthesized through an intermediate such as isobutyric acid or 2-methyl-1,3-propanediol (Karsten Lang, Katja Buehler and Andreas Schmid, Multistep Synthesis of (S)-3-Hydroxyisobutyric acid from glucose using Pseudomonas taiwanensis VLB120 B83 T7 catalytic biofilms, Advanced Synthesis & Catalysis, 357(8), 1919-1927 (2015)).

However, the present inventors have identified an enzyme that converts methylmalonyl-CoA directly to methylmalonate semialdehyde, that is, an enzyme having methylmalonyl-CoA reductase (MMCR) activity, among MCR enzymes. The use of the identified enzyme enables 3-HIBA to be produced through a short metabolic pathway.

Herein, the identified enzyme may be kingdom Archaea-derived methylmalonyl-CoA reductase. The present inventors have screened various enzymes from malonyl-CoA reductase (MCR) enzymes which use substrates different from a substrate for MMCR but are functionally similar to MMCR. Among the screened enzymes, an enzyme that can also use methylmalonyl-CoA as a substrate was selected. In addition, the changes in amounts of NADH and NADPH, which are used as cofactors, after the reaction with methylmalonyl-CoA, were measured by absorbance, and an enzyme that is reactive with methylmalonyl-CoA was selected.

As a result, in an embodiment, the enzyme that is reactive with methylmalonyl-CoA used as a reaction substrate may be, for example, methylmalonyl-CoA reductase derived from one or more Archaea species selected from the group consisting of Candidatus Caldiarchaeum subterraneum, Sulfolobales archaeon Acd1 and Sulfolobus acidocaldarius Ron12/I.

In the present invention, the enzyme that is reactive with methylmalonyl-CoA used as a reaction substrate was sequenced. As a result, it could be found that the enzyme comprises a sequence that is at least 60% homologous or at least 80% similar to a sequence of SEQ ID NO: 23.

Based on this finding, in an embodiment, the enzyme may comprises a sequence having a sequence homology of at least 60%, at least 65%, at least 70%, at least 75%, at least 80%, at least 85%, at least 90%, at least 92%, at least 93%, at least 95%, at least 97%, at least 98%, at least 99% or 100% to the sequence represented by SEQ ID NO: 23.

In another embodiment, the enzyme may comprises a sequence having a sequence similarity of at least 80%, at least 85%, at least 90%, at least 92%, at least 93%, at least 95%, at least 97%, at least 98%, at least 99% or 100% to the sequence represented by SEQ ID NO: 23.

As used herein, the term “homology” refers to the percent identity between two amino acid or polynucleotide moieties for comparison. The term “similarity” refers to the degree to which two amino acid or polynucleotide sequences are functionally or structurally identical to each other as determined by the comparison window. The sequence homology or similarity can be determined by comparing sequences using the standard software, for example, a program called BLASTN or BLASTX, developed based on BLAST (Proc. Natl. Acad. Sci. USA, 90, 5873-5877, 1993).

The methylmalonyl-CoA reductase may comprise a sequence having a sequence homology of at least 60%, at least 65%, at least 70%, at least 75%, at least 80%, preferably, at least 90%, at least 92%, at least 93%, at least 95%, at least 97%, at least 98%, at least 99% or 100% to the sequence represented by at least one selected from the group consisting of SEQ ID NOs: 3 to 5.

In some cases, methylmalonyl-CoA reductase according to the present invention may also be mutated using a known technique known in the art in order to increase the efficiency of production of 3-HIBA.

In another aspect, the present invention is directed to a mutant microorganism having the ability to produce methylmethacrylic acid, which contains, in addition to the above-described genes (i) to (iv), (v) a gene encoding 3-hydroxyisobutyrate dehyrotase. The 3-hydroxyisobutyrate dehyrotase is an enzyme that is involved in the conversion of 3-HIBA to methylmethacrylic acid.

The sources and sequences of genes encoding methylmalonyl-CoA mutase, methylmalonyl-CoA epimerase and 3-hydroxyisobutyrate dehydrogenase, in addition to the enzyme methylmalonyl-CoA reductase used in the present invention, are shown in Table 1 below.

TABLE 1
Enzyme candidates that are involved in conversion to 3-HIBA

| | Gene | | | SEQ ID |
|---|---|---|---|---|
| Enzyme | name | Sequence ID | Source | NO: |
| methylmalonyl- | MCM | Msed_0638 | Metallosphaera | 7, 8 |
| CoA mutase | | Msed_2055 | sedula | |
| methylmalonyl- | MCE | Msed_0639 | Metallosphaera | 9 |
| CoA epimerase | | | sedula | |
| hydroxyisobutyrate | 3- | G_9075 | Pseudomonas | 10 |
| dehydrogenase | HIBADH | | putida | |

As used herein, the term “microorganism” may include any organism included in the domain of Archaea, bacteria or eukaryotes, and may include any kind of prokaryotes or eukaryotes, for example, Archaea, bacteria, yeasts or fungi. For example, the microorganism that is used in the present invention may be E. coli, S. cerevisiae, C. blankii, or C. rugosa.

The gene encoding the enzyme is exogenous. The term “exogenous” means that the gene encoding Archaea-derived MMCR (methylmalonyl-CoA reductase) is introduced into the host microorganism. The introduction can be achieved by introducing the MMCR-encoding gene into the genetic material of the host microorganism by insertion into a material such as a plasmid, that is, a chromosomal or non-chromosomal genetic material.

Examples of a carbon source that may be used in the present invention include carbohydrates such as glucose, fructose, sucrose, lactose, maltose, starch and cellulose, fats such as soybean oil, regular sunflower oil, castor oil and coconut oil, fatty acids such as palmitic acid, stearic acid and linoleic acid, alcohols such as glycerol and ethanol, and organic acids such as acetic acid. These carbon sources may be used alone or in combination.

In still another aspect, the present invention is directed to a method for producing 3-HIBA, comprising a step of culturing the above-described mutant microorganism. In addition, the present invention is directed to a method for producing methylmethacrylic acid, comprising a step of culturing the above-described mutant microorganism.

The mutant microorganism may be cultured according to a known method at a temperature of 20-45° C. in the presence of a carbon source. As the carbon source that is used in the culture, the following carbon sources may be used alone or in combination: (i) carbohydrates, including monosaccharides, for example, glucose, sucrose, lactose, fructose, maltose, molasses, starch or cellulose; (ii) oils and fats, for example, soybean oil, regular sunflower oil, peanut oil or coconut oil; (iii) fatty acids, for example, palmitic acid, stearic acid and linoleic acid; (iv) alcohols, for example, glycerol or methanol; (v) amino acids, for example, L-glutamate or L-valine; and (vi) organic acids, for example, acetic acid. In some cases, the culture medium may include a known nitrogen source, a phosphorus source, a metal salt required for growth, a precursor, or a pH adjusting agent.

The 3-HIBA or methylmethacrylic acid expressed by culture of the mutant microorganism may be separated and recovered. For example, the expressed 3-HIBA or methylmethacrylic acid may be separated from the culture medium by passing the culture medium through a filter or using a centrifuge or a sedimentation device. In addition, pure 3-HIBA or methylmethacrylic acid may be recovered using an additional osmosis or purification method.

### EXAMPLES

Hereinafter, the present invention will be described in further detail with reference to examples. It will be obvious to a person having ordinary skill in the art that these examples are illustrative purposes only and are not to be construed to limit the scope of the present invention. Thus, the substantial scope of the present invention will be defined by the appended claims and equivalents thereof.

### Example 1

### Screening of MMCR

Using Uniprot, various enzymes were screened from MCR (malonyl-CoA reductase) enzymes that use a substrate different for a substrate for MMCR but are functionally similar to MMCR in that they can convert malonyl-CoA to malonate semialdehyde. Among the screened enzymes, enzymes that can also use methylmalonyl-CoA as a substrate were selected, and the products of the enzymes were examined, thereby screening MMCR (methylmalonyl-CoA reductase). As MMCR candidates, enzymes having similar protein sequences were selected by searching Sulfolobus tokodaii-derived MCR enzymes having known structures through BLAST. Selection of enzymes was performed using Uniprot according to the above-described procedure, and as a result, two MCR enzymes and four aspartate-semialdehyde dehydrogenase enzymes, which have MMCR activity, were obtained (Table 2).

TABLE 2
MMCR candidates

| | | | | SEQ |
|---|---|---|---|---|
| Gene | | | | ID |
| name | Sequence ID | Organism | Enzyme | NO: |
| MCRst | Q96YK1 | Sulfolobus | Malonyl-CoA | 1 |
| | | tokodaii | reductase | |
| | | (strain DSM | | |
| | | 16993) | | |
| MCRms | A4YEN2 | Metallosphaera | Malonyl-CoA | 2 |
| | | sedula | reductase | |
| | | (strain ATCC | | |
| | | 51363) | | |
| ASDcc | E6N613_9ARCH | Candidatus | Aspartate- | 3 |
| | | Caldiarchaeum | semialdehyde | |
| | | subterraneum | dehydrogenase | |
| ASDsar | GI:519043079 | Sulfolobales | Aspartate- | 4 |
| | | archaeon | semialdehyde | |
| | | Acd1 | dehydrogenase | |
| ASDsac1 | M1IGV1_9CREN | Sulfolobus | Aspartate- | 5 |
| | | acidocaldarius | semialdehyde | |
| | | Ron12/I | dehydrogenase | |
| ASDsac2 | M1J171_9CREN | Sulfolobus | Aspartate- | 6 |
| | | acidocaldarius | semialdehyde | |
| | | Ron12/I | dehydrogenase | |

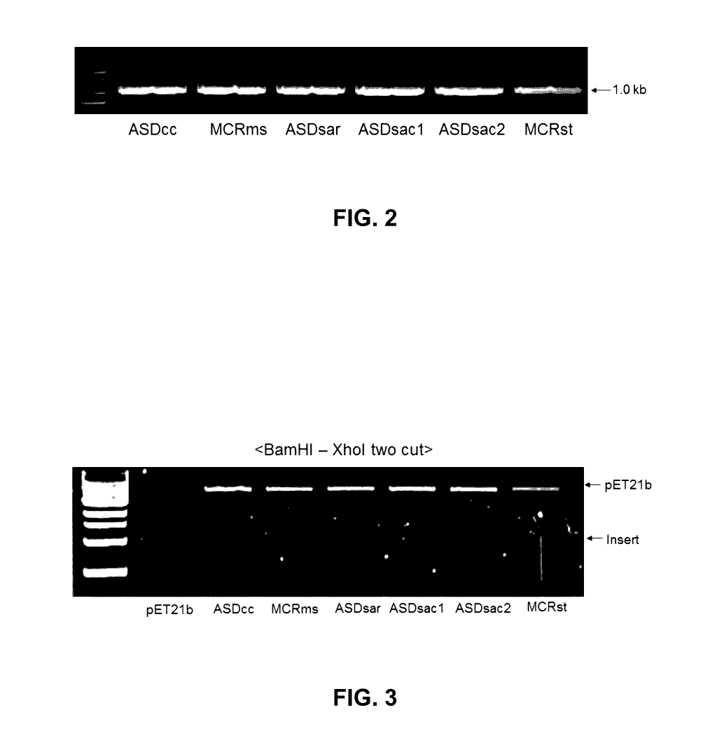

The MMCR candidate genes shown in Table 2 above were cloned by PCR using the synthesized primers shown in Table 3 below under the following conditions: 30 cycles, each consisting of 10 sec at 98° C., 5 sec at 55° C. and 1 min at 72° C. The results of the PCR are shown in FIG. 2. Next, each of the PCR products was inserted into a pET21b vector using T4 DNA ligase (FIG. 3). As shown in FIG. 3, the desired constructs were made. The constructs were transformed into the expression strain E. coli BL21(DE3), thereby constructing strains.

TABLE 3
Primer sequences

| Gene | Primer sequence | SEQ ID NO: |
|---|---|---|
| MCRstF | ATGAGCTCATGAGAAGAACTTTGAAA | 11 |
| MCRstR | TTCTCGAGTTACTTTTCGATGTAACC | 12 |
| MCRmsF | ATGAGCTCATGAGAAGAACTTTGAAA | 13 |
| MCRmsR | TTCTCGAGTTATCTCTTATCAATGTA | 14 |
| ASDccF | ATGAGCTCATGAAAACTTACTCCGTC | 15 |
| ASDccR | TTCTCGAGTCATTCGCCTAACAACCA | 16 |
| ASDsarF | ATGAGCTCATGAGAAGAACTTTGAAG | 17 |
| ASDsarR | TTCTCGAGTTACTTAGGGATGTAACC | 18 |
| ASDsac1F | ATGACGTCATGATAAGAGTCTTGAAA | 19 |
| ASDsac1R | TTCTCGAGTCAATCCATGTAACCCTT | 20 |
| ASDsac2F | ATGAGTCATGAGAAGAGTTTACAAA | 21 |
| ASDsac2R | TTCTCGAGTCAGATGTACTTTCTGTT | 22 |

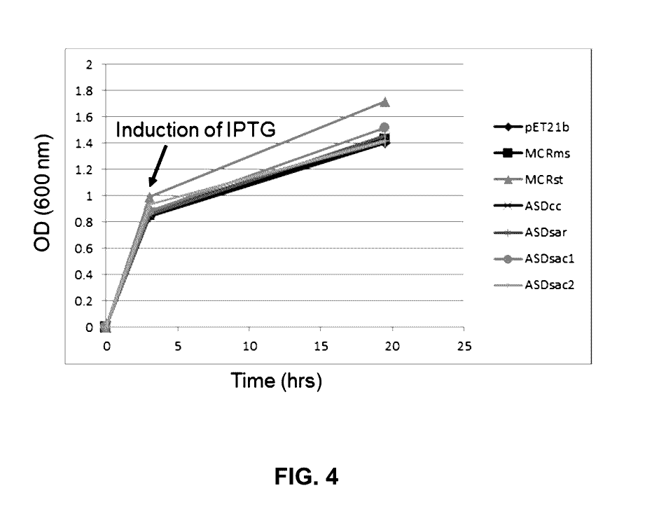

Each of the constructed strains (transformants) was cultured in LBA medium at 37° C. and 200 rpm. When the OD value at 600 nm reached 0.5-0.8, 1 mM of isopropyl-1-thio-β-D-galactopyranoside (IPTG) was added to the medium, and then each strain was cultured overnight 16° C., thereby expressing an each of the MMCR enzymes shown in Table 2 above. The results of the expression are shown in FIG. 4.

Each of the cultured strains was centrifuged to remove the supernatant, and then the cells were collected and lysed by a sonicator, followed by centrifugation to collect the supernatant, thereby preparing enzyme solutions. The concentration of the enzyme in each enzyme solution was quantitatively analyzed by performing color development using a Pierce BCA kit at 37° C. for 30 minutes and then measuring the absorbance at 562 nm using a microplate spectrophotometer. The results of the analysis are shown in Table 4 below.

TABLE 4
Results of quantitative analysis of protein
concentrations in cultured transformant cells

| Sample | A562 | Con.(ug/ml) | Dilution Factor (*5) |
|---|---|---|---|
| Pet21b | 0.659 | 573.674 | 2868.37 |
| ASDcc | 0.636 | 548.696 | 2743.48 |
| MCRms | 0.682 | 598.652 | 2993.26 |
| ASDsar | 0.742 | 663.812 | 3319.06 |
| ASDsac1 | 0.741 | 662.726 | 3313.63 |
| ASDsac2 | 0.699 | 617.114 | 3085.57 |
| MCRst | 0.718 | 637.748 | 3188.74 |

Each of the obtained enzyme solutions was allowed to react with methylmalonyl-CoA as a substrate in a medium containing the components shown in Table 5 below, and then changes in the amounts of NADH and NADPH, which are used as cofactors, were analyzed by measuring the absorbance at 365 nm with a microplate spectrophotometer, thereby determining whether the enzymes would be reactive with methylmalonyl-CoA.

TABLE 5

| Reagent | Stock Cone. | | Working Con. | | Volume(μl) |
|---|---|---|---|---|---|
| Tris-HCL(Ph 7) | 100 | mM | 50 | mM | 100 |
| NAD(P)H | 4 | mM | 0.4 | mM | 20 |
| MgCl₂ | 10 | mM | 2 | mM | 40 |
| Cell extract | 2x dlution | | | | 20 |
| Methylmalonyl- | 3.0 | mM* | 0.3 | mM | 20 |
| CoA | | | | | |

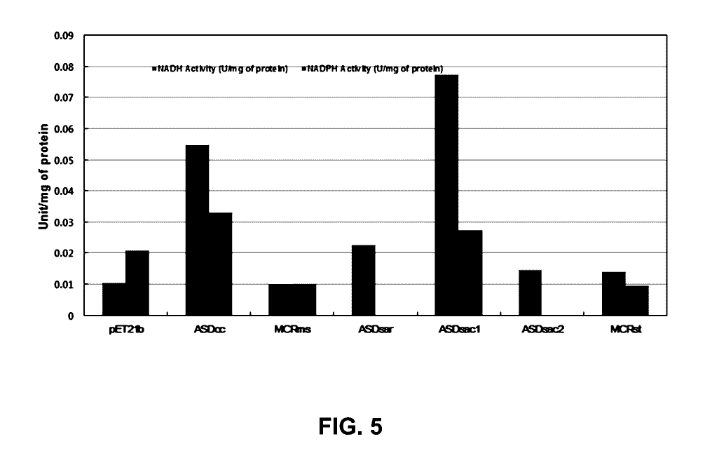

The rate of consumption of NADH and NADPH versus the amount of protein used was calculated as activity. As a result, it could be seen that, when NADH was used as a cofactor, ASDcc, ASDsar and ASDsac1 showed reactivity with methylmalonyl-CoA (FIG. 5).

### Example 2

### Production of Methylmalonate Semialdehyde

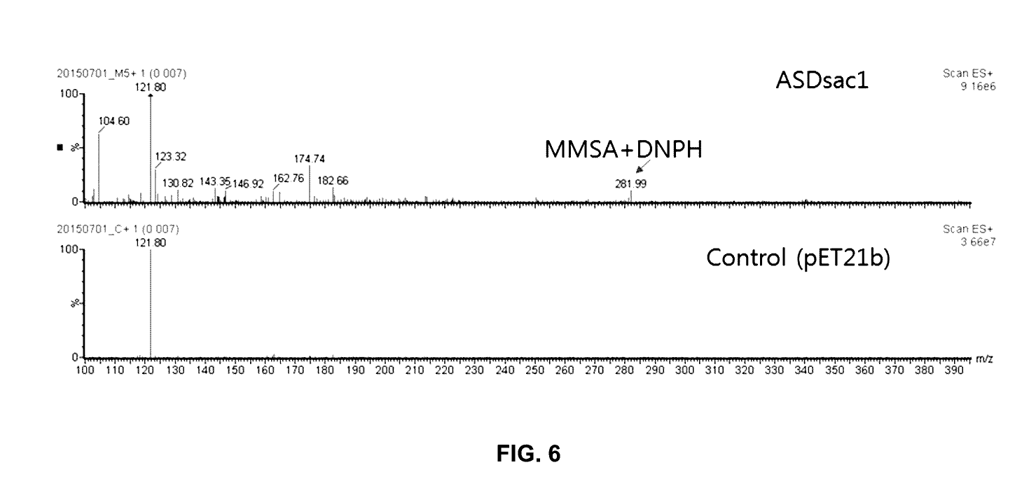

In order to examine the methylmalonate semialdehyde productivity of ASDsac1 showing the best performance among the primarily selected enzymes, ASDsac1 was allowed to react with 0.5 g/L of methylmalonyl-CoA, and the reaction product was analyzed by MS. As a result, as shown in FIG. 6, it was shown in the reaction product of ASDsac1 that methylmalonate semialdehyde was produced.

### Example 3

### Construction of 3-HIBA-Producing E. coli Strain

ASDsac1 confirmed to produce methylmalonate semialdehyde was optimized using a codon optimization tool (http://sg.idtdna.com/CodonOpt) so as to optimize the expression thereof in E. coli, and was used to construct an E. coli strain that produces 3-HIBA as shown in FIG. 1 (see Table 6).

The E. coli 3-HIBA-producing pathway genes shown in Table 6 below were cloned by PCR using the primers shown in Table 7 below under the following conditions: 30 cycles, each consisting of 10 sec at 98° C., 5 sec at 55° C. and 1 min at 72° C. Each of the PCR products was inserted into pET21b and pET26b vectors using T4 DNA ligase. After construction of the desired constructs was confirmed, each of the constructs was transformed into the expression strain E. coli BL21 (DE3), thereby constructing strains.

TABLE 6
Design of 3-HIBA-producing E. coli strains containing ASDsac1 gene

| | pET21b (Amp^{R}) | | | | |
|---|---|---|---|---|---|
| | | | methylmalonyl- | pET26b(Kan^{R}) | |
| | methylmalonyl- | methylmalonyl- | CoA | | 3-hydroxyisobutyrate |
| | CoA mutase α | CoA mutase β | epimerase | | dehydrogenase |
| | (EC: 5.4.99.2) | (EC: 5.4.99.2) | (EC: 5.1.99.1) | MMCR | (EC 1.1.1.31) |
| 1 | Control (Only vectors) | | | | |
| 2 | E. coli | | P. freudenreichii | S. acidocaldarius | E. coli |
| | (GI:42945) | | (GI:22022367) | | (GI:331077966) |
| 3 | P. freudenreichii | P. freudenreichii | P. freudenreichii | S. acidocaldarius | E. coli |
| | (GI:45834) | (GI:581476) | (GI:22022367) | | (GI:331077966) |

TABLE 7
Primers for cloning of E. coli 3-HIBA-producing
pathway genes

| Gene | Primer sequence |
|---|---|
| F ASDsac1 | AGCT GAGCTC ATGCGTCGCGTTCTGAAAGCAGCGA |
| | (SEQ ID NO: 24) |
| R ASDsac1 | TCGA CTCGAG TCAATCCATATAACCCTTCTCCACA |
| | (SEQ ID NO: 25) |
| F PME | CTA GCTAGC |
| | ATGAGTAATGAGGATCTTTTCATCTGTATCG |
| | (SEQ ID NO: 26) |
| R PME | CCG CTCGAG TCAGTTCTTCGGGTACTGGGTG |
| | (SEQ ID NO: 27) |
| F PMMa | CCC AAGCTT ATGAGCACTCTGCCCCGTTTTG |
| | (SEQ ID NO: 28) |
| R PMMa | ATAAGAAT GCGGCCGC CTAGGCATCGAGCGAAGCCC |
| | (SEQ ID NO: 29) |
| F PMMb | CCC AAGCTT ATGAGCAGCACGGATCAGGGG |
| | (SEQ ID NO: 30) |
| R PMMb | ATAAGAAT GCGGCCGC |
| | TCACTTCGCGACTCCCAAGATATC |
| | (SEQ ID NO: 31) |
| F EMM | AT GAGCTC ATGTCTAACGTGCAGGA |
| | (SEQ ID NO: 32) |
| R EMM | CCG CTCGAG ATCATGATGCTGGCTTATCAGATTCAG |
| | (SEQ ID NO: 33) |
| F HIBADH | AT GAGCTC ATGAAAACGGGATCTGA |
| | (SEQ ID NO: 34) |
| R HIBADH | TT CTCGAG TCATGATTTCGCTCCCG |
| | (SEQ ID NO: 35) |
| BglII T7 | GGA AGATCT CAAAAAACCCCTCAAGACCCGTTTA |
| Ter | (SEQ ID NO: 36) |
| EcoNI T7 | GCATT CCTGCATTAGG |
| Pro | TTAATACGACTCACTATAGGGGAATTGTG |
| | (SEQ ID NO: 37) |
| SgrAI T7 | CCGG CACCGGCG CAAAAAACCCCTCAAGACCCGTTTA |
| Ter | (SEQ ID NO: 38) |
| SphI T7 | CATG GCATGC |
| Pro | TTAATACGACTCACTATAGGGGAATTGTG |
| | (SEQ ID NO: 39) |
| SphI T7 | CATG GCATGC CAAAAAACCCCTCAAGACCCGTTTA |
| Ter | (SEQ ID NO: 40) |
| BglII T7 | GGA AGATCT TTAATACGACTCACTATAGGGGAATTGTG |
| Pro | (SEQ ID NO: 41) |

### Example 4

### Production and Fermentation of 3-HIBA in E. coli

For culture of the recombinant E. coli strains constructed in Example 3, 30 ml 2×M9 (Na₂HPO₄-2H₂O, KH₂PO₄, NaCl, NH₄Cl) minimal medium was placed in a 250-ml flask, and glucose (10 g/L), 600 ul of 100× trace metal solution (5 g/L EDTA, 0.83 g/L FeCl₃-6H₂O, 84 mg/L ZnCl₂, 13 mg/L CuCl₂-2H₂O, 10 mg/L CoCl₂-2H₂O, 10 mg/L H₃BO₃, 1.6 mg/L MnCl₂-4 H₂O), 60 ug/l of vitamin 12, 1 mg/ml of biotin, 1 mg/ml of thiamin, 0.25 g/L of MgSo₄, 50 ug/ml of kanamycin, and 100 ug/ml of ampicillin were added thereto. Next, each of the strains was cultured in the medium in an incubator at 37° C., and when the optical density (OD) reached 0.8, expression of the 3-HIBA-producing gene was induced by 0.1 mM IPTG.

After addition of IPTG, the culture temperature was changed to 30° C., and 5 g/L of sodium succinate as a substrate for 3-HIBA production was further added to the medium, after which additional culture was performed at 200 rpm for 72 hours. At 72 hours of the culture, a portion of the culture product was collected to measure the optical density (OD) and the production of 3-HIBA.

### Example 5

### Analysis of 3-HIBA

The culture product obtained by 72 hours of culture in Example 4 was centrifuged (4° C. and 13,000 rpm for 10 min) to remove the cells. 40 ml of the cell-free supernatant sample was dried for 124 hours by a freeze drying method using a freeze dryer (ilShinBioBase, Korea), and then dissolved in 2 ml of distilled water, thereby preparing 3 ml of an about 13-fold concentrated HPLC sample.

Using an Agilent 1200 HPLC system (Agilent, USA) having an injection volume of 10 liters, the sample was analyzed. In the HPLC, the Hypercarb column (150 mm×4.6 mm) (Thermo, USA) was kept at 30° C., and DIW (0.1% sulfuric acid) and CAN (0.1% sulfuric acid) were used as a mobile phase at a flow rate of 1 ml/min. In addition, a DAD detector (Agilent, USA) was used for analysis.

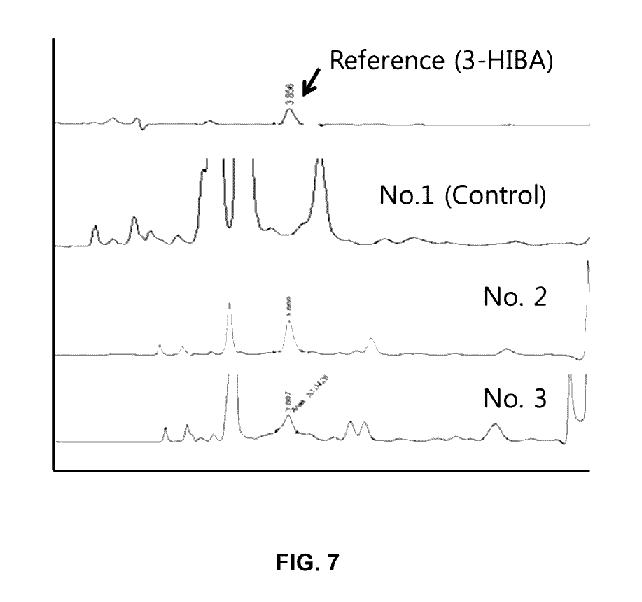

As shown in FIG. 7, the analysis results indicated that, when strains 2 and 3 constructed in Example 3 were cultured for hours and concentrated 13-fold, 3-HIBA was produced in amounts of 69 ppm and 21 ppm.

Although the present invention has been described in detail with reference to the specific features, it will be apparent to those skilled in the art that this description is only for a preferred embodiment and does not limit the scope of the present invention. Thus, the substantial scope of the present invention will be defined by the appended claims and equivalents thereof.

1. A mutant microorganism derived from a microorganism having the ability to produce succinyl-CoA from a carbon source, wherein the mutant microorganism contains genes encoding the following enzymes and has the ability to produce 3-HIBA (3-hydroxyisobutyric acid):
(i) methylmalonyl-CoA mutase;
(ii) methylmalonyl-CoA epimerase;
(iii) methylmalonyl-CoA reductase; and
(iv) 3-hydroxyisobutyrate dehydrogenase, wherein the enzyme of (iii) is an enzyme exhibiting methylmalonyl-CoA reductase activity among enzymes having malonyl-CoA reductase activity.

2. The mutant microorganism of claim 1, wherein enzyme (iii) is a monofunctional enzyme exhibiting methylmalonyl-CoA reductase activity and conversion activity methylmalonyl-CoA to methylmalonate semialdehyde, selected from among enzymes having malonyl-CoA reductase activity.

3. The mutant microorganism of claim 1, wherein the enzyme exhibiting methylmalonyl-CoA reductase activity among enzymes having malonyl-CoA reductase activity is derived from an organism in kingdom Archae.

4. The mutant microorganism of claim 3, wherein enzyme (iii) is derived from one or more Archaea species selected from the group consisting of Candidatus Caldiarchaeum subterraneum, Sulfolobales archaeon Acd1, and Sulfolobus acidocaldarius Ron12/I.

5. The mutant microorganism of claim 1, wherein enzyme (iii) comprises a sequence having a sequence homology of at least 60% SEQ ID NO: 23.

6. The mutant microorganism of claim 1, wherein the enzyme of (iii) comprises a sequence having a sequence homology of at least 60% to the sequence represented by any one selected from the group consisting of SEQ ID NO: 3, SEQ ID NO: 4, and SEQ ID NO: 5.

7. The mutant microorganism of claim 1, wherein the enzyme of (iii) comprises any sequence among sequences selected from the group consisting of SEQ ID NO: 3, SEQ ID NO: 4, and SEQ ID NO: 5.

8. A method for producing 3-HIBA, comprising the steps of:
culturing the mutant microorganism of claim 1 to produce 3-HIBA; and
recovering the produced 3-HIBA.
